(19) United States
(12) Patent Application Publication
(10) Pub. No.: US 2017/0056683 A1
(43) Pub. Date: Mar. 2, 2017

(54) METHOD AND DEVICE USING HIGH-INTENSITY MILLIMETER WAVES

(71) Applicant: ARIEL-UNIVERSITY RESEARCH AND DEVELOPMENT COMPANY LTD., Ariel (IL)

(72) Inventors: Stella Aronov, Petach Tikva (IL); Asher Yahalom, Givat Shmuel (IL); Jacob Levitan, Herzliya (IL); Konstantin Komoshvili, Bat Yam (IL)

(73) Assignee: ARIEL-UNIVERSITY RESEARCH AND DEVELOPMENT COMPANY LTD., Ariel (IL)

(21) Appl. No.: 15/139,353

(22) Filed: Apr. 27, 2016

Related U.S. Application Data
(60) Provisional application No. 62/152,979, filed on Apr. 27, 2015.

(51) Int. Cl. A61N 5/00

(52) U.S. Cl. CPC A61N 5/00

(57) ABSTRACT

Disclosed are methods and devices using millimeter waves, that in some embodiments are useful for treating cancers such as lung cancers.

### RELATED APPLICATION

The present application gains priority from U.S. Provisional Patent Application 62/152,979 filed 27 Apr. 2015, which is included by reference as if fully set-forth herein.

### FIELD AND BACKGROUND OF THE INVENTION

The invention relates to methods and devices using irradiation of living cells with high-intensity millimeter waves for treatment, such as millimeter waves having an intensity of not less than about 0.2 W/cm² (1 W), that in some embodiments are useful for treating cancers such as lung cancers.

Low-intensity (of up to 10 mW/cm² or less) millimeter waves having wavelengths of between 40 GHz and 70 GHz are used in the field of medicine for the treatment of various diseases, especially in former USSR nations, see for example: Pakhomov et al. IEEE Transactions on Plasma Science 2000, 28(1); Betskii et al. Critical Reviews in Biomedical Engineering 2000, 28(1,2), 247-268 and Betskii et al. “Millimeter Waves in Biology and Medicine”, III-IRE, 26-30 Oct. 2009 (in Russian).

### SUMMARY OF THE INVENTION

The invention relates to methods and devices using irradiation of living cells of an organism with high-intensity millimeter waves for treatment, such as millimeter waves having an intensity of not less than about 0.2 W/cm², that in some embodiments are useful for treating cancers such as lung cancers.

In some embodiments of the devices and methods, the treatment is the treatment of cancer, and the irradiated cells are cancerous cells. In some such embodiments, the treatment of the cancerous cells with the millimeter waves leads to weakening and/or death of the cancerous cells.

According to an aspect of some embodiments of the present invention, there is provided a device useful for treatment, the device configured to direct millimeter waves at living cells to irradiate the cells with millimeter waves having an intensity of not less than about 0.2 W/cm², thereby treating the cells. In some embodiments, the device is useful for the treatment of cancer and the irradiated cells are cancerous cells.

In some embodiments, the device comprises a millimeter-wave generation component configured to generate the millimeter waves as at least one beam of millimeter waves. In some embodiments, the millimeter-wave generation component comprises at least one member of the group consisting of:

at least one free electron laser/maser for the generation of the millimeter waves; and

at least one gyrotron for the generation of the millimeter waves.

In some embodiments, the device is configured for in vivo the irradiating of the cells.

In some embodiments, the device comprises a component configured to identify cancerous cells marked with a marker.

In some embodiments, the device comprises guiding components suitable for directing millimeter waves as desired. In some embodiments, the guiding components are integrated with or in a medical device. In some embodiments, the medical device is selected from the group consisting of a catheter, an endoscope, a bronchoscope, an ophthalmoscope and a fundus camera.

According to an aspect of some embodiments of the present invention, there is also provided a method for the treatment of a subject in need thereof, comprising:

providing millimeter waves having an intensity of not less than about 0.2 W/cm²; and

irradiating living cells of the subject with at least one dose of the millimeter waves, thereby treating the cells.

In some embodiments, the treatment is the treatment of cancer, and the irradiated cells are cancerous cells.

In some embodiments of the method, the millimeter waves are provided as at least one beam of millimeter waves.

In some embodiments of the method, the millimeter waves are generated by at least one member of the group consisting of:

at least one free electron laser/maser for the generation of the millimeter waves; and

at least one gyrotron for the generation of the millimeter waves.

The duration of a single dose of the millimeter waves is any suitable duration. In some embodiments of the method, a duration of a single dose of the millimeter waves is not more than about 1 second, not more than about 100 milliseconds, not more than about 10 milliseconds, not more than about 1 millisecond, not more than about 100 microseconds, not more than about 30 microseconds and even not more than about 10 microseconds. In some embodiments, a duration of a single dose of the millimeter waves is not less than about 1 nanosecond, not less than about 10 nanoseconds, not less than about 0.1 microseconds and even not less than about 0.9 microseconds. In some embodiments, the duration of a single dose of the millimeter waves is not less than about 0.9 microseconds and not more than about 10 microseconds.

In some embodiments, a single dose of the millimeter waves is a pulse of millimeter waves having a substantially continuous intensity.

Any suitable frequency of administering a dose of the millimeter waves may be used. In some embodiments, the irradiating of the cells is with not less than one dose of the millimeter waves in a month and in some embodiments not less than one the dose of the millimeter waves in a week.

The intensity of the millimeter waves is any suitable intensity. In some embodiments of the method or the device, the intensity of the millimeter waves is not less than about 2 W/cm², 0 not less than about 20 W/cm². and even not less than about 200 W/cm². In some embodiments of the method or the device, the intensity is not more than about 2000 W/cm².

The frequency of the millimeter waves is any suitable frequency. In some embodiments, the millimeter waves comprise electromagnetic radiation having a frequency of between about 30 GHz and about 300 GHz, and in some embodiments having a frequency of between about 75 GHz and about 110 GHz.

Additional aspects and embodiments of the invention are described in the specification hereinbelow and in the appended claims.

Some aspects of the invention may be understood with reference to the article “Millimeter Waves Non-Thermal Effect on Human Lung Cancer Cells” published at the 3^{rd} International IEEE Conference on Microwave, Communications, Antennas and Electronic Systems—COMCAS 2011, Tel Aviv, November 7-9 (2011) by some of the Inventors.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate+/−10%.

Embodiments of methods and/or devices of the teachings herein may involve performing or completing selected tasks manually, automatically, or a combination thereof. Some embodiments of the teachings herein are implemented with the use of components that comprise hardware, software, firmware or combinations thereof. In some embodiments, some components are general-purpose components such as general purpose computers or oscilloscopes. In some embodiments, some components are dedicated or custom components such as circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1A is a bar graph showing the effect of irradiation on mortality of cancerous H1299 cells and non-cancerous MCF-10A cells 2 hours after irradiation according to an embodiment of the teachings herein; and

FIG. 1B is a bar graph showing the effect of irradiation on mortality of cancerous H1299 cells and non-cancerous MCF-10A cells 7 days hours after irradiation according to an embodiment of the teachings herein.

### DESCRIPTION OF SOME EMBODIMENTS OF THE INVENTION

The invention relates to methods and devices using irradiation of living cells of an organism with high-intensity millimeter waves for treatment, such as millimeter waves having an intensity of not less than about 0.2 W/cm², that in some embodiments are useful for treating cancers such as lung cancers.

Interaction of biological cells with low-intensity (10 mW/cm² or less) millimeter wavelength electromagnetic radiation has been studied for several decades, see, e.g., the review Apollonio et al. “Feasibility for Microwaves Energy to Affect Biological Systems Via Nonthermal Mechanisms: A Systematic Approach” in IEEE Transactions on Microwave Theory and Techniques 2013, 61(5) 2031-2045. Millimeter waves effect cells thermally and non-thermally.

The thermal effect of low-intensity millimeter waves is the direct heating of intra- and/or extracellular water. Heating cells to temperatures of up to 50-60° C. destroys cancerous as well as non-cancerous cells.

Non-thermal effects of low-intensity millimeter waves have been studied using different in vitro biological models, such as bacteria and mammalian cell lines as well as in vivo models. Despite the research efforts, the mechanism of non-thermal effects of millimeter waves on biological cells has not yet been fully elucidated. Different millimeter waves frequencies used in vitro to study these effects show significant changes in protein, RNA and DNA expression as well as significant changes in tissue, cells and cell compartments such as membranes, cytoskeletons, chromosomes and nuclei.

Herein is disclosed treatment of organisms and cells using methods and devices that use high-intensity (i.e., not less than about 0.2 W/cm²) millimeter waves, for example in the treatment of cancer (e.g., lung cancer) by irradiation of cancerous cells (e.g., lung cancer cells) with the high-intensity millimeter waves.

The application of high-intensity millimeter waves in accordance with the teachings herein was studied in cells derived from adenocarcinoma of human lung cancer patients (H1299) and in healthy cells (MCF-10A).

Human lung cancer cells (H1299) were irradiated with high-intensity W-band (75-100 GHz) millimeter waves in vitro to identify effects suitable for clinical treatment.

The results show that irradiation of the H1299 cancer cells for 4 microseconds at 400 W/cm² or 1000 W/cm² leads to an at least four-fold increase in mortality compared to non-irradiated controls. The increased mortality was not observed in the non-tumorigenic MCF-10A epithelial cells.

The principles, uses and implementations of the teachings of the invention may be better understood with reference to the accompanying description and figures. Upon perusal of the description and figures present herein, one skilled in the art is able to implement the teachings of the invention without undue effort or experimentation.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

### Method

According to an aspect of some embodiments of the invention, there is provided a method for the treatment (in some embodiments, of cancer) in a subject in need thereof, comprising:
providing millimeter waves having an intensity of not less than about 0.2 W/cm²; and
irradiating living cells (in some embodiments, cancerous (i.e., cancer) cells) of the subject with at least one dose of the millimeter waves.
   In some embodiments, the subject is in vitro cells. In some embodiments, the cells are in vivo cells. In some embodiments the subject is a human. In some embodiments, the subject is a non-human animal.

In some embodiments, the millimeter waves are provided as at least one beam of millimeter waves. In some embodiments, the millimeter waves are provided as a single beam of millimeter waves. In some embodiments, the millimeter waves are provided as at least two beams of millimeter waves.

In some embodiments, the millimeter waves are generated by at least one member of the group consisting of: at least free electron laser/maser for the generation of the millimeter waves; and at least one gyrotron for the generation of the millimeter waves.

### Treated Cells

In some embodiments, the irradiated cells are cancer cells, that is to say, a group of cells (e.g., making up tissue of an organism) is irradiated in accordance with the teachings herein such that irradiated cancer cells of the tissue are harmed, e.g., (killed, damaged and/or weakened) to an extent greater than non-cancer cells of the tissue are harmed.

In some embodiments, the irradiation of the cancer cells is performed concurrently with the administration of a chemotherapeutic agent. In some such embodiments, the cancer cells are harmed by being less resistance to the effects of the chemotherapeutic agent, rendering the concurrent treatment with the chemotherapeutic agent more effective and/or less toxic (by allowing a reduced dose to be administered).

In some embodiments, the subject is a living organism, the need is that the subject is suffering from a cancer susceptible to treatment according to the teachings herein. A cancer susceptible to treatment according to the teachings herein is a cancer which irradiation of cancerous cells of the cancer in accordance with the teachings herein harms the cancerous cells to a clinically-relevant degree.

A subject suffering from any such cancer may be treated in accordance with the teachings herein. In some embodiments, the cancer is a cancer selected from the group consisting of skin, melanoma, retinal, uterine, lung, non small cell lung carcinoma, oral, brain, bladder, esophageal, stomach, liver, pancreas, thyroid, colon, kidney, ovary, breast, colon, prostate, blood and leukemia.

Any suitable type of cancerous cell may be irradiated in accordance with the teachings herein. In some embodiments, the cancer cell is a melanocyte, a free melanocyte, an epithelial melanocyte, an epithelial cell, a neuroendocrine cell, a squamous cell, a mesenchymal cell and a lympoblast.

Any suitable type of tumor may be irradiated in accordance with the teachings herein. In some embodiments, the tumor is selected from the group consisting of melanoma, retinoblastoma, adenocarcinoma, carcinoma, neuroblastoma, sarcoma and teratosarcoma.

### Intensity of Millimeter Waves

As noted above, some embodiments of the teachings herein comprise irradiating cells with millimeter waves at an intensity not less than about 0.2 W/cm². In some embodiments, the intensity is not less than about 2 W/cm². In some embodiments, the intensity is not less than about 20 W/cm². In some embodiments, the intensity is not less than about 200 W/cm². In some embodiments, the intensity is not less than about 400 W/cm².

In some embodiments, the intensity is not more than about 2000 W/cm². In some embodiments, the intensity is not more than about 1600 W/cm².

### Frequency of Millimeter Waves

Millimeter waves having any suitable frequency may be used in implementing the teachings herein.

In some embodiments, the millimeter waves comprise electromagnetic radiation having a frequency of between about 30 GHz and about 300 GHz, in some embodiments a frequency of between about 75 GHz and about 110 GHz, between about 90 GHz and about 105 GHz, between about 97 GHz and about 104 GHz and even between about 100 GHz and about 102 GHz.

In some embodiments, the millimeter waves comprise electromagnetic radiation having a frequency of about 101 GHz.

### Dose of Millimeter Waves

As noted above, some embodiments of the teachings herein involve irradiating cells of a subject with at least one dose of millimeter waves, in some embodiments, cancerous cells. Typically, the exact number of doses sufficient for a course of treatment is determined for a specific subject by a treating health-care professional and often involves administration of one or more doses, followed by examination after a time to see the effect of the administration, optionally followed by administration of additional one or more doses.

In some embodiments, the duration of a single dose of the millimeter waves is not more than about 1 second, not more than about 100 milliseconds, not more than about 10 milliseconds, not more than about 1 millisecond. not more than about 100 microseconds, not more than about 30 microseconds, not more than about 10 microseconds, and even not more than about 1 microseconds.

In some embodiments, the duration of a single dose of the millimeter waves is not less than about 1 nanosecond, not less than about 10 nanoseconds, not less than about 0.1 microseconds, and even not less than about 0.9 microseconds.

In some embodiments, the duration of a single dose of the millimeter waves is not less than about 0.9 microseconds and not more than about 10 microseconds, e.g., about 1, about 2, about 3, about 4 or even about 5 microseconds.

In some embodiments, a single dose of the millimeter waves is a pulse of millimeter waves having a substantially continuous intensity.

In some embodiments, the irradiating of the cells is with not less than one dose of the millimeter waves in a month, In some embodiments, the irradiating of the cells is with not less than one dose of the millimeter waves in a week.

The method according to the teachings herein may be implemented using any suitable device or combination of devices. That said, in some embodiments it is preferred to use a device according to the teachings herein.

### Device Useful for Treatment Using Millimeter Waves

According to an aspect of some embodiments of the teachings herein, there is also provided a device useful for treatment (in some embodiments of cancer), the device configured to direct millimeter waves at living cells (in some embodiments, cancerous cells) to irradiate the cells at an intensity of not less than about 0.2 W/cm², thereby treating the cells. In some embodiments, the device is configured to irradiate the cells in vivo.

In some embodiments, the device comprises a millimeter-wave generation component configured to generate the millimeter waves as at least one beam of millimeter waves. In some embodiments, the millimeter-wave generation component comprises at least one member of the group consisting of: at least one free electron laser/maser for the generation of the millimeter waves; and at least one gyrotron for the generation of the millimeter waves.

In some embodiments, the device and components thereof are configured to be suitable for use in a medical setting, for example, enclosed in a casing that can be cleaned and/or sterilized at a level sufficient for a medical setting.

In some embodiments, the device comprises aiming and/or guiding components suitable for directing the millimeter waves as desired (e.g., for irradiation in vivo) preferably under the control of medical personnel or suitably-configured computer (e.g., includes a component configured to identify cancerous cells marked with a marker, e.g., a radioactive or fluorescent marker). Depending on the embodiment, guiding components typically include aiming optics, waveguides, reflectors and focussers, and are often integrated with or in medical devices such as catheters, endoscopes (e.g., bronchoscopes), ophthalmoscopes and fundus cameras. For example, in some such embodiments, the device includes a guiding component that is a flexible wave guide, in some such embodiments having an internal diameter of not more than 3 mm, allowing a generated beam to travel through the medical device to treat affected tissue. Additionally or alternatively, in some embodiments, the device includes a guiding components that is a reflector to direct a generated beam into the body of the medical device, or to direct a generated past a portion of the body of the medical device that does not allow a curved beam-path (e.g., a discontinuous 90° turn). Additionally or alternatively, in some embodiments, the distal end of the device includes a beam spreader, a beam focusser and/or a beam straightener, allowing the intensity of the beam to be changed and/or the amount of tissue irradiated to be changed by changing the cross-sectional size of the beam that impinges on the tissue to be changed.

In some embodiments, the device includes a visible light source that has substantially the same cross sectional size or substantially the same beam divergence or both substantially the same cross sectional size and beam divergence as a generated beam of millimeter waves emerging from the distal end. Such a visible light source allows a person using the device to illuminate an area of tissue with visible light prior to and/or simultaneously with irradiation with millimeter waves in order to visualize the tissue that will be irradiated with the millimeter waves.

In some embodiments, the device is configured to generate millimeter waves having the intensity as described above with reference to the method according to the teachings herein.

In some embodiments, the device is configured to generate millimeter waves having the frequency as described above with reference to the method according to the teachings herein.

In some embodiments, the device is configured to generate millimeter waves for durations corresponding to the doses described above with reference to the method according to the teachings herein.

### EXAMPLES

### Example 1

### Effect of High-Intensity Irradiation on Cancerous and Healthy Cells In Vitro

### Millimeter Waves

Millimeter waves in accordance with the teachings herein were provided using a Free Electron Laser (FEL) located at Ariel University that can produce millimeter waves having a tunable frequency in the range 95-105 GHz with a tunable output power of 0.1 kW to 10 kW and described in A. Gover, A. Faingersh, A. Eliran, M. Volshonok, H. Kleinman, S. Wolowelsky, B. Kapilevich, Y. Lasser, Z. Seidov, M. Kanter, A. Zinigrad, M. Einat, Yu. Lurie, A. Abramovich, A. Yahalom, Y. Pinhasi, E. Weisman & J. Shiloh “Radiation Measurements in the New Tandem Accelerator FEL” Nuclear Instruments & Methods A 528/1-2 pp. 23-27 (2004). The millimeter waves generated by the FEL were directed to a pyramidal horn antenna (SGH-10 by Millitech Inc., Northampton, Mass., USA) that was aimed upwards. Since the rectangular aperture of the antenna was 2 cm×2.5 cm the intensity of the millimeter waves emitted by the antenna could be varied from about 20 W/cm² (0.1 kW/5 cm²) to about 2000 W/cm² (10 kW/5 cm²).

### Media and Cell Cultures

Human lung cancer cells (H1299, also known as NCI-H1299 or CRL-5803) were generously provided by Professor Uri Alon of the Weizmann Institute. Cells were grown in RPMI medium (Biological Industries, Beth Ha'emek, Israel) supplemented with: 10% fetal bovine serum (FBS, from Sigma), penicillin 100 Units/ml (from Sigma), streptomycin 100 μg/ml (from Sigma); and 1 mM glutamine (from Biological Industries, Beth Ha'emek, Israel).

MCF-10A cells of a non-tumorigenic epithelial cell line were generously provided by Professor Yossi Shaul of the Weizmann Institute, Rehovot. MCF-10A is commonly recognized as a “normal” breast epithelial cell line derived from the breast tissue of a 36-year-old patient with fibrocystic changes. These cells exhibit numerous features of normal breast epithelium, including lack of tumorigenicity in nude mice, lack of anchorage-independent growth, and dependence on growth factors and hormones for proliferation and survival. The MCF-10A cells were grown in DMEM/F12 including: 5% horse serum (HS, from Sigma), penicillin 100 Units/ml (from Sigma), streptomycin 100 μg/ml (from Sigma), EGF 20 μg/ml (from Peprotech), hydrocortisone 0.5 □g/ml (from Sigma), cholera toxin 100 μg/ml (from Sigma), and insulin 10 mg/ml (from Sigma).

The appropriate medium was seeded with the respective cells a day before the experiment to proliferate. Both types of cells (cancerous HL299 and control MCF-10A) were cultured at 37° C. in 5% CO₂. Under these conditions, the proliferation rate of both types of cells was similar (12 to 14 hours doubling time).

### Irradiation of the Cancerous and Non-Cancerous Cells

To irradiate the living cells, Petri dishes (3.5 cm diameter, Nalge Nunc International, Penfield, N.Y., USA) containing cells to be irradiated having about 30% confluency in a suitable medium were removed from an incubator and placed to rest on the upper surface of the antenna. The intensity was selected (either 400 W/cm² or 1000 W/cm² and the FEL activated to generate a single pulse of millimeter wave radiation having a frequency of 101 GHz having a substantially continuous intensity for about 4 microseconds. Since the Petri dishes had a 3.5 cm diameter, each irradiated Petri dish contained both irradiated and non-irradiated cells. Subsequent to irradiation, the Petri dishes with the now-irradiated cells were placed back in the incubator.

Control Petri dishes were removed from the incubator and placed to rest on the upper surface of the antenna, and then placed back in the incubator without activating the FEL so that the cells were not irradiated but had otherwise undergone the same interruption in incubation as the irradiated cells.

### Evaluation of the Effect of Irradiation on the Cells

A first set of dishes was removed from the incubator about 2 hours after irradiation and second set of dishes was removed from the incubator 7 days after irradiation. Each one of the two sets of dishes included some of the dishes with H1299 lung cancer cells that had been irradiated, some of the dishes with MCF-10A non-cancerous cells that had been irradiated, some of the dishes with H1299 lung cancer cells that had not been irradiated and some of the dishes with MCF-10A non-cancerous cells that had not been irradiated. The cells in the dishes were washed once with 2 ml of PBS, fixed with a 4% paraformaldehyde solution at room temperature for 30 minutes, and stained with 50 microgram/ml of 4′,6-diamidino-2-phenylindole (DAPI) at 4° C. for 10 minutes. The thus-fixed and stained cells were washed with PBS. Fluorescent images of the cells were acquired in the usual way through a microscope (Nikon Instruments Inc., Melville, N.Y., USA with an objective ×20 using an emission filter of 408-480 nm for DAPI staining) and analysed using ImageJ and Photoshop software (Adobe Systems Incorporated, Mountain View, Calif., USA). At least 400 cells in each sample that showed DNA accumulation and fragmentation were counted. The percentage of dead cells was calculated.

The death rate of the cancerous H1299 cells that had been irradiated was 40%-67% (after both 2 hours and after 7 days) while the death rate of the cancerous H1299 cells that had not been irradiated was 8%-10% (after both 2 hours and after 7 days)

The death rate of the non-cancerous MCF-10A cells, irradiated or not, after 2 hours or after 7 days, was 3%-10%.

### Results

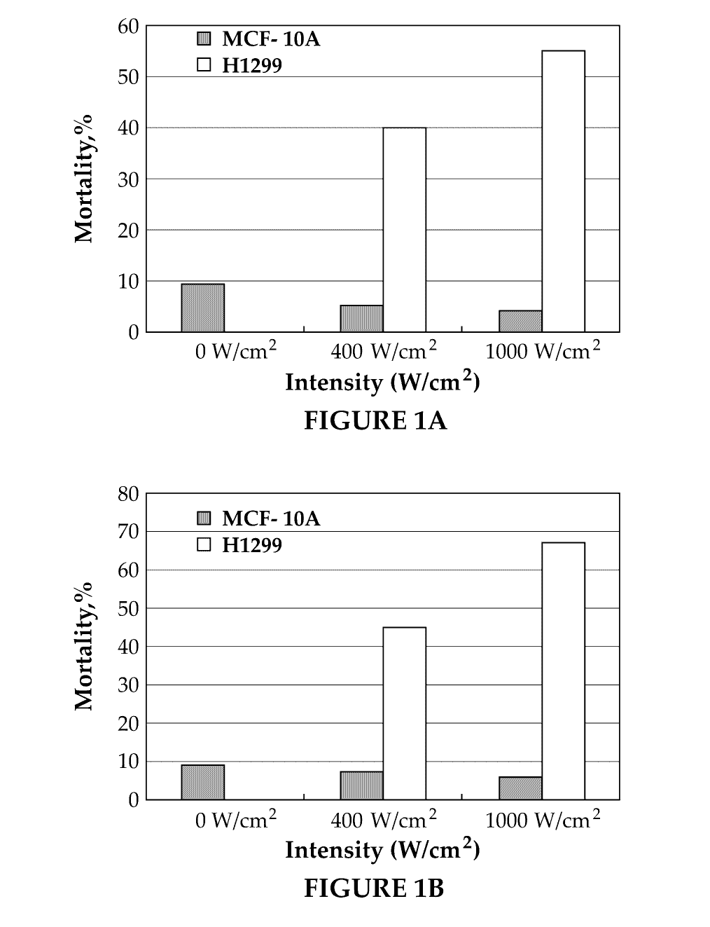

Table 1 and FIGS. 1A and 1B show the death rate of H1299 human lung cancer cells and non-cancerous MCF-10A cells, calculated as the percentage of dead cells in the total population following irradiation at 400 W/cm² or 1000 W/cm², as compared to control (non-irradiated) cells of the same type. As shown in the table and the Figures, a significant increase in mortality was seen in the irradiated cancerous cells on day 1 and day 7, but no increase in mortality was seen in the non-cancerous cells.

TABLE 1

| | 0 W/cm² | 400 W/cm² | 1000 W/cm² |
|---|---|---|---|
| | (2 hour/7 days) | (2 hour/7 days) | (2 hour/7 days) |
| Control H1299 | 10%/8% | | |
| | (n = 2350) | | |
| Irradiated H1299 | — | 40%/45% | 55%/67% |
| | | (n = 465) | (n = 576) |
| Control MCF-10A | 9.3%/8% | | |
| | (n = 2850) | | |
| Irradiated MCF-10A | | 5.1%/7.3% | 4.1%/3.4% |
| | | (n = 2300) | (n = 2520) |

### Summary and Discussion

The human lung cancer (H1299) cells derived from an adenocarcinoma of a patient (with lack of p53 functional protein) were irradiated by a single 4 microsecond pulse of high-intensity 101 GHz millimeter waves. A substantial proportion of the cancerous H1299 cells died as a result of the irradiation. Non-cancerous MCF-10A cells irradiated under the same conditions did not show increased mortality.

### Further Examples

The above experiments are performed with cells from additional cancer cell lines listed in Table 2, all being commercially available, e.g., from Creative Bioarray (Shirley, N.Y., USA) or ATCC (Manassas, Va., USA). Details of some of the cell lines are available from the suppliers or from the Centro Biotecnologie Avanzate, Genova, Italy.

TABLE 2

| organ | cell line | cell type | tumor type |
|---|---|---|---|
| skin (melanoma) | MEL-HO | free melanocyte | melanoma |
| retinal | Y-79 | epithelial | retinoblastoma |
| | | melanocyte | |
| cervical | SISO | epithelial | adenocarcinoma |
| uterine | AN3-CA | epithelial | adenocarcinoma |
| lung (NSCLC) | CAL-12T | epithelial | adenocarcinoma |
| oral | RPMI-2650 | epithelial | carcinoma |
| brain | LAN-2 | neuroendocrine | neuroblastoma |
| bladder | CLS-439 | epithelial | carcinoma |
| esophageal | KYSE-30 | epithelial | carcinoma |
| | | (squamous) | |
| stomach | MKN-45 | epithelial | adenocarcinoma |
| liver | HEP-3B | epithelial | carcinoma |
| (hepatocellular) | | | |
| pancreas | PA-TU-8902 | epithelial | adenocarcinoma |
| thyroid | S-117 | mesenchymal | sarcoma |
| colon | HT29 | epithelial | adenocarcinoma |
| kidney | ACHN | epithelial | adenocarcinoma |
| ovary | OVCAR-3 | epithelial | adenocarcinoma |
| ovary | PA-1 | epithelial | teratocarcinoma |
| breast | MCF-7 | epithelial | adenocarcinoma |
| breast - mdr | MDA-MB231 | epithelial | adenocarcinoma |
| colon | HCT 116 | epithelial | carcinoma |
| prostate | PC3 | epithelial | adenocarcinoma |
| blood (leukemia) | J45.01 | lymphoblast | |
| | | (acute T cell | |
| | | leukemia) | |

In some embodiments, the teachings herein are applicable to any type of cancer. In some preferred embodiments, the teachings herein are particularly applicable to treating adenocarcinomas, that is to say cancers of epithelial cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

1. A device useful for treatment, the device configured to direct millimeter waves at living cells to irradiate the cells with millimeter waves having an intensity of not less than about 0.2 W/cm², thereby treating the cells.

2. The device of claim 1, said device comprising a millimeter-wave generation component configured to generate said millimeter waves as at least one beam of millimeter waves.

3. The device of claim 2, wherein said millimeter-wave generation component comprises at least one member of the group consisting of:
at least one free electron laser/maser for the generation of said millimeter waves; and
at least one gyrotron for the generation of said millimeter waves.

4. The device of claim 1, wherein the device is configured for in vivo said irradiating of the cells.

5. The device of claim 1, comprising a component configured to identify cancerous cells marked with a marker.

6. The device of claim 1, comprising guiding components suitable for directing millimeter waves as desired.

7. The device of claim 6, said guiding components integrated with or in a medical device.

8. The device of claim 7, said medical device selected from the group consisting of a catheter, an endoscope, a bronchoscope, an ophthalmoscope and a fundus camera.

9. The device of claim 1, wherein said intensity is not less than about 2 W/cm².

10. The device of claim 1, wherein said millimeter waves comprise electromagnetic radiation having a frequency of between about 30 GHz and about 300 GHz.

11. A method for the treatment of a subject in need thereof, comprising:
providing millimeter waves having an intensity of not less than about 0.2 W/cm²; and
irradiating living cells of the subject with at least one dose of said millimeter waves, thereby treating the cells.

12. The method of claim 11, wherein said millimeter waves are provided as at least one beam of millimeter waves.

13. The method of claim 11, wherein said intensity is not less than about 2 W/cm².

14. The method of claim 11, wherein said millimeter waves comprise electromagnetic radiation having a frequency of between about 30 GHz and about 300 GHz.

15. The method of claim 11, wherein a duration of a single said dose of said millimeter waves is not more than about 1 second.

16. The method of claim 11, wherein a duration of a single said dose of said millimeter waves is not more than about 100 milliseconds.

17. The method of claim 11, wherein a duration of a single said dose of said millimeter waves is not less than about 1 nanosecond.

18. The method of claim 11, wherein a duration of a single said dose of said millimeter waves is not less than about 10 nanoseconds.

19. The method of claim 11, wherein a single said dose of said millimeter waves is a pulse of millimeter waves having a substantially continuous intensity.

20. The method of claim 11, wherein said treatment is the treatment of cancer, and said cells are cancerous cells.
